(19) United States
(12) Patent Application Publication
(10) Pub. No.: US 2010/0125220 A1
(43) Pub. Date: May 20, 2010

(54) SURGICAL METHOD FOR GASTROCNEMIUS MUSCLE REDUCTION

(76) Inventor: Yeon Jae SEONG, Seoul (KR)

Correspondence Address: NIXON & VANDERHYE, PC, 901 NORTH GLEBE ROAD, 11TH FLOOR, ARLINGTON, VA, 22203, US

(21) Appl. No.: 12/401,803

(22) Filed: Mar. 11, 2009

(30) Foreign Application Priority Data
Nov. 14, 2008 (KR) 10-2008-0113260

(51) Int. Cl. A61B 5/0488; A61N 1/05; A61M 5/00

(52) U.S. Cl. 600/546; 607/40; 604/20

(57) ABSTRACT

A surgical method for gastrocnemius muscle reduction is provided. The method may search for a motor nerve associated with a gastrocnemius muscle of a patient, inserts a probe by penetrating an epidermis around the motor nerve and locates a tip of the probe in the motor nerve, stimulates the motor nerve through supply of an electric signal to the probe, thereby shutting down the motor nerve.

### CROSS-REFERENCE TO RELATED APPLICATION

This application claims the benefit of Korean Patent Application No. 10-2008-0113280, filed on Nov. 14, 2008, in the Korean Intellectual Property Office, the disclosure of which is incorporated herein by reference.

### BACKGROUND OF THE INVENTION

1. Field of the Invention

The following embodiments relate to a surgical method for gastrocnemius muscle reduction, and more particularly, to a surgical method for gastrocnemius muscle reduction through shutting down a motor nerve associated with the gastrocnemius muscle.

2. Description of Related Art

The human nervous system is used for transmission/reception of signals. Paths for neural signal delivery are generated by neural signals delivering command signals which cause motions and sensory information such as a pain, heat, cool air, and a sense of touch.

A sensory nerve is a nerve where a sensory cell deliver an impact to a central nerve occurring after an external stimulus is delivered to sensory organs, and is referred to as a afferent nerve. Occasionally, signals delivered to the central nerve may be abnormal. As examples of the abnormal signals, there is compression of a nerve which causes a constriction of a minor nerve or a referred pain. The abnormal signals may bring about pains, seizures, distortions, cramps, and the like. Therefore, shutting down the paths for signal delivery associated with the abnormal signals may relieve pain.

In addition, a motor nerve is a peripheral nerve, which controls somatomotor, is connected to a skeletal muscle, and is a basic nerve which delivers a stimulus occurring in a center of a nervous system. When the paths for signal delivery associated with a motor nerve are shut down, muscles are paralyzed, and the paralyzed muscle may be atrophied. As an example of the atrophy of the muscle, there may be a case of thinning of muscle.

### BRIEF SUMMARY

According to an aspect of the present invention, there is provided a surgical method for gastrocnemius muscle reduction including: detecting nerve line information associated with a gastrocnemius muscle of a patient using a nerve scan apparatus; searching for a motor nerve associated with the gastrocnemius muscle based on the nerve line information; inserting a probe by penetrating an epidermis around the motor nerve and locating a tip of the probe in the motor nerve; and shutting down the motor nerve by stimulating the motor nerve through supply of an electric signal to the probe.

In an aspect of the present invention, the motor nerve is a tibial nerve.

Also, in an aspect of the present invention, the gastrocnemius muscle may include a medial gastrocnemius muscle and a lateral gastrocnemius muscle.

Also, in an aspect of the present invention, the motor verve may be either a nerve to a medial gastrocnemius muscle branched from the tibial nerve or a nerve to a lateral gastrocnemius muscle. The searching for the motor nerve associated with the gastrocnemius muscle based on the nerve line information searches for a branch point of the tibial nerve and a common peroneal nerve, and searches for a nerve to the medial gastrocnemius muscle and a nerve to the lateral gastrocnemius muscle, branched from the tibial nerve.

In an aspect of the present invention, the nerve scan apparatus may be an electromyogram (EMG) apparatus.

Also, in an aspect of the present invention, the probe may consist of an insulator which is coated on a surgical steel and coated on a surface of the surgical steel, and the tip of the probe may not be coated by the insulator.

Also, in an aspect of the present invention, the surgical steel may include tungsten, and the insulator may be a rubber.

Also, in an aspect of the present invention, the electrical signal may be transmitted to a return electrode associated with the probe via the patient.

Also, in an aspect of the present invention, shutting down of the motor nerve may stimulate the motor nerve through supplying the electric signal to the probe and by applying a dissolving agent on the motor nerve using a needle, so that the motor nerve may be shut down.

Also, in an aspect of the present invention, the searching for the motor nerve associated with the gastrocnemius muscle based on the nerve line information may search for the motor nerve associated with the gastrocnemius muscle using the probe.

An aspect of the present invention provides a surgical method for gastrocnemius muscle reduction which may be more secure and easy to use by shutting down a motor nerve without incision.

An aspect of the present invention provides a surgical method for gastrocnemius muscle reduction which may minimize a feeling of uneasiness after operation and a possibility of recurrence by shutting down using both an electric signal and a resolving agent.

An aspect of the present invention provides a surgical method for gastrocnemius muscle reduction which may minimize damages of nerves and other muscles by searching for a nerve which is directly associated to the gastrocnemius muscle.

An aspect of the present invention provides a surgical method for gastrocnemius muscle reduction which may prevent stiffness of a muscle and maintain a portion of functions of a motor nerve by shutting down the motor nerve instead of resection of the motor nerve.

Additional aspects, features, and/or advantages of the invention will be set forth in part in the description which follows and, in part, will be apparent from the description, or may be learned by practice of the invention.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a flowchart illustrating a surgical method for gastrocnemius muscle reduction according to example embodiments;

FIG. 2 is a diagram illustrating an example of a nerve scan apparatus and a nerve line of a patient's gastrocnemius muscle according to example embodiments;

FIG. 3 is a diagram illustrating a tibial nerve, a nerve to a medial gastrocnemius muscle, a nerve to a lateral gastrocnemius muscle, and a common peroneal nerve according to example embodiments;

FIG. 4 is a diagram illustrating an example of a probe according to example embodiments; and

FIG. 5 is a diagram illustrating an operation of shutting down of a motor nerve according to example embodiments.

### DETAILED DESCRIPTION OF EMBODIMENTS

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

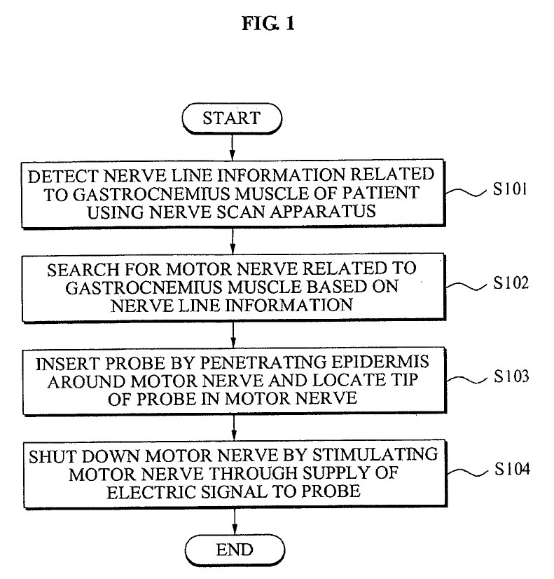

FIG. 1 is a flowchart illustrating a surgical method for gastrocnemius muscle reduction according to example embodiments.

As illustrated in FIG. 1, the surgical method for gastrocnemius muscle reduction according to example embodiments may include operations S101 through S104.

In operation S101, nerve line information associated with a gastrocnemius muscle of a patient may be detected via a nerve scan apparatus. Specifically, a practitioner may detect the nerve line information associated with the gastrocnemius muscle of the patient using the nerve scan apparatus. In this instance, the nerve scan apparatus may include an electromyogram (EMG) apparatus. The EMG apparatus records electrical signals coming from a brain or a muscle by simulating nerves of the patient and verifies whether there is an abnormality of the electric signals. As an example of the examination of the EMG apparatus, there are a needle electromyography (needle EMG) testing electrical activities from a muscular fiber by stimulating a muscle with a needle, a nerve conduction study (NCS) testing a nerve function by applying combined potential caused by supplying an electrical stimulus on a peripheral nerve, a repetitive nerve stimulation test (RNS) testing a degree of an amplitude decrease of combined muscles action potential, and the like.

Also, the gastrocnemius muscle is an aponeurosis forming an achilles tendon, along with a soleus muscle. The gastrocnemius muscle affects plantar flexion of a foot, and helps a genus muscle to flex when the genus muscle is not supporting a weight. The gastrocnemius muscle prevents dorsiflexion of an ankle joint when the foot is fixed on a ground supporting the weight. In addition to this, the gastrocnemius muscle may maintain an extension state of a knee joint supporting the weight. The gastrocnemius muscle includes a medial gastrocnemius muscle and a lateral gastrocnemius muscle.

The nerve line information is connected with the gastrocnemius muscle to include information about peripheral nerves which control somatomotor of the gastrocnemius muscle. As examples of the peripheral nerves which control somatomotor of the gastrocnemius muscle, there are a tibial nerve, a nerve to the medial gastrocnemius muscle, and a nerve to the lateral gastrocnemius muscle, which are branched from the tibial nerve. According to the example embodiments, the nerve line information may be stored in a storage apparatus in the EMG apparatus or may be exposed through an exposure apparatus included in the EMG apparatus.

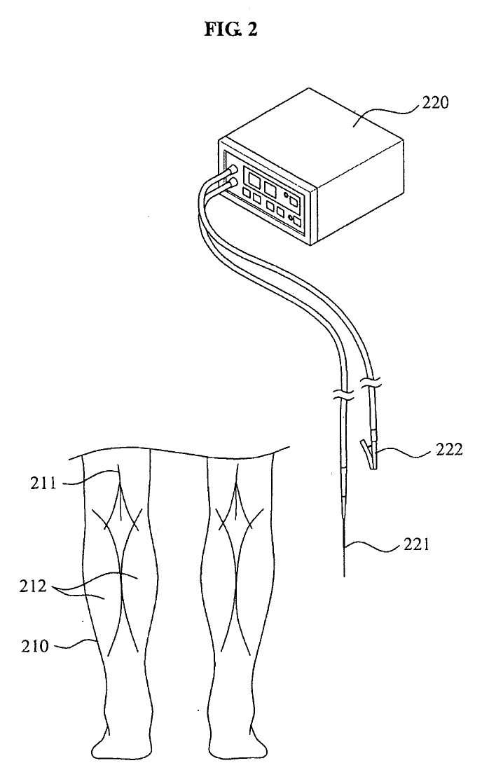

FIG. 2 is a diagram illustrating an example of a nerve scan apparatus and a nerve line of a patient's gastrocnemius muscle according to example embodiments.

Referring to FIG. 2, a practitioner may detect nerve line information associated with a gastrocnemius muscle 212 of a patient 210 using an electromyogram (EMG) apparatus 220. As an example, the practitioner supplies a stimulus to a motor nerve associated with the gastrocnemius muscle 212 of the patient 210 using probes 221 and 222 and measures electrical activities coming from the gastrocnemius muscle 212, thereby detecting information about a nerve line 211.

In operation S102, a motor nerve associated with the gastrocnemius muscle 212 may be detected based on the nerve line information. Specifically, the practitioner may search for the motor nerve associated with the gastrocnemius muscle 212 based on the nerve line information.

According to the example embodiments, the motor nerve may be a tibial nerve. That is, the practitioner may search for a tibial nerve associated with the gastrocnemius muscle 212 based on the nerve line information in operation S102.

Also, according to the example embodiments, the motor nerve may be either a medial gastrocnemius muscle or a lateral gastrocnemius muscle, branched from the tibial nerve. That is, the practitioner may search for either the medial gastrocnemius muscle or the lateral gastrocnemius muscle, associated with the gastrocnemius muscle 212 based on the nerve line information. Consequently, the practitioner may search for at least one of the tibial nerve associated with somatomotor of the medial gastrocnemius muscle and the lateral gastrocnemius muscle, a nerve to the medial gastrocnemius muscle, and a nerve to the lateral gastrocnemius muscle.

According to the example embodiments, the practitioner may search for a branch point of the tibial nerve and a common peroneal nerve, and search for the nerve to the medial gastrocnemius muscle and the nerve to the lateral gastrocnemius muscle, branched from the tibial nerve. In this instance, the branch point of the tibial nerve and the common peroneal nerve may be used for accurately identifying the common peroneal nerve which is responsible for ankle movement of the patient. If the practitioner shuts down the common peroneal nerve in operation S104, a problem may occur in ankle movement.

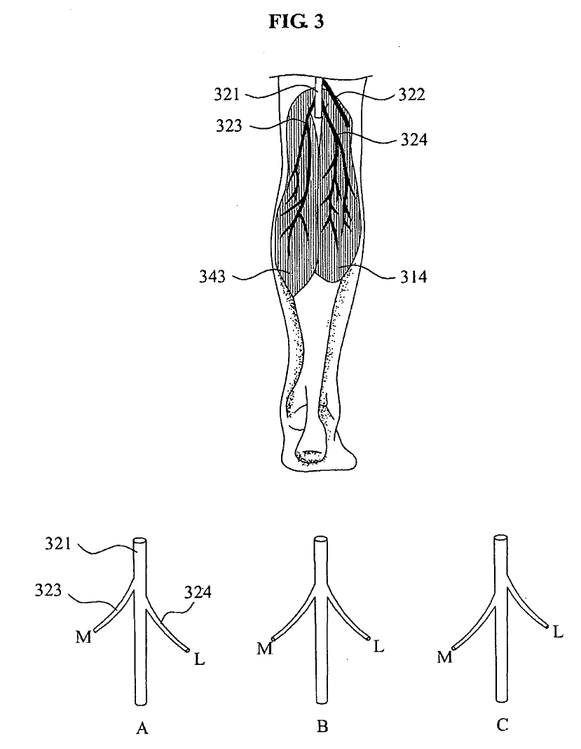

FIG. 3 is a diagram illustrating a tibial nerve, a nerve to a medial gastrocnemius muscle, a nerve to a lateral gastrocnemius muscle, and a common peroneal nerve according to example embodiments.

As illustrated in FIG. 3, the nerve 323 to the medial gastrocnemius muscle which is a motor nerve responsible for a medial gastrocnemius muscle 313 of the patient and the nerve 324 to the lateral gastrocnemius muscle which is a motor nerve responsible for a lateral gastrocnemius muscle 314 are branched from a tibial nerve 321. Shapes of the tibial nerve 321, the nerve 323 to the medial gastrocnemius muscle and the nerve 324 to the lateral gastrocnemius muscle may vary depending on patients, and, as an example of the various shapes, A, B and C may be included as illustrated in FIG. 3. Also, the common peroneal nerve 322 is branched from the tibial nerve 321.

In operation S103, the practitioner may insert a probe by penetrating an epidermis around the motor nerve and may locate a tip of the probe in the motor nerve. Specifically, the practitioner may locate the tip of the probe in the motor nerve by penetrating the epidermis with the probe without creating an incision of the epidermis around the motor nerve. Here, the motor nerve may be one of the tibial nerve, the nerve to the medial gastrocnemius muscle, and the nerve to the lateral gastrocnemius muscle. Also, the probe may be an apparatus for penetration which is made of surgical steel. Except for the tip of the probe, a surface of the surgical steel may be coated with an insulator. The surgical steel may include tungsten.

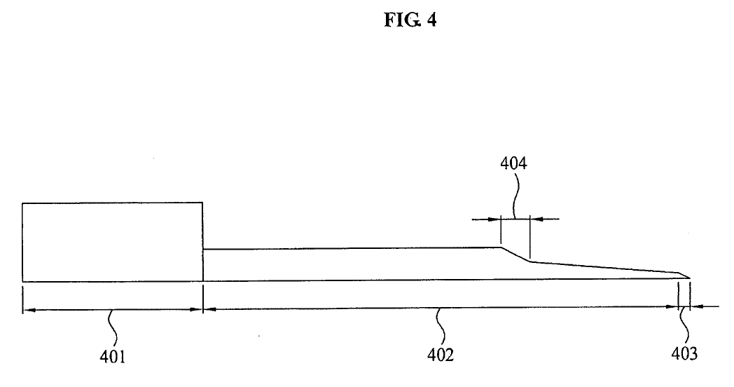

FIG. 4 is a diagram illustrating an example of a probe according to example embodiments.

As illustrated in FIG. 4, the probe may consist of a handle 401, a body 402, and a tip 403. The body 402 is coated with an insulator, and a rubber and insulation chemicals may be included in the insulator. The tip 403 and a portion 404 of the body 402 may be configured in a double bevel for smooth penetration through an epidermis. The handle 401 may be connected with a power supply device, and the power supply device may pass through the body 402 to transmit an electric signal via the tip 403 of the probe.

According to the example embodiments, the practitioner may search for the motor nerve associated with the gastrocnemius muscle using the probe based on the nerve line information in operation S102. Specifically, the practitioner may perform a first search for the motor nerve based on the nerve line information, and may perform a second search for the motor nerve using the probe. In this instance, the second search process may include a process of stimulating the motor nerve by penetrating an epidermis with the probe, and a process of searching for the motor nerve with reference to degrees of compression or laxity of the gastrocnemius muscle which corresponds to the stimulating of the motor nerve.

In operation S104, the practitioner may shut down the motor nerve by stimulating the motor nerve through supply of an electric signal to the probe. Here, the electric signal may include an intermediate frequency current or a high frequency current. After the electric signal shuts down the motor nerve, the electrical signal may be transmitted to a return electrode associated with the probe via the patient. The return electrode may be formed in a broad curved surface shape so as to be resistantly or capacitively combined with a body of the patient. Through this, a heat occurring in the return electrode may be scattered to be vanished through the broad curved surface.

According to the example embodiments, the practitioner may cause atrophy in the gastrocnemius muscle associated with the motor nerve by permanently or temporarily paralyzing a predetermined percentage of functions of the motor nerve. In this instance, the atrophy of the gastrocnemius muscle may appear in a type of thinning of the gastrocnemius muscle, and the patient may expect a slimmer calf through the atrophy. According to the example embodiments, the predetermined percentage may be at least seventy percent, and the patient may go on with life as usual with remaining thirty percent of the functions of the gastrocnemius muscle. Here, a soleus muscle of the patient may compensate for the functions of the gastrocnemius muscle for the usual life.

Also, the practitioner may reduce the gastrocnemius muscle more easily and securely by shutting down the motor nerve using the probe. When the practitioner directly incises the epidermis and the motor nerve, stiffness of a muscle and expansion of the incised portion may occur. In addition, since the nerve to the lateral gastrocnemius muscle is located deeper than the nerve to the medial gastrocnemius muscle, the incision of the nerve to the lateral gastrocnemius muscle is difficult process.

According to example embodiments, the practitioner may shut down the motor nerve by stimulating the motor nerve through supplying an electric signal to the probe and by applying a dissolving agent on the motor nerve using a needle. Here, the dissolving agent may include ethanol or phenol. According to the example embodiments, the ethanol may be 90% of ethanol.

Specifically, the practitioner may shut down the motor nerve at a first stage by stimulating the motor nerve using the supplied electric signal to the probe, and may shut down the motor nerve at a second stage by applying the dissolving agent on the motor nerve using the needle. In this instance, the needle may separately exist from the probe, however the needle may be included inside the probe or may externally exist depending on the example embodiments.

As described the above, a surgical method for gastrocnemius muscle reduction may reduce a gastrocnemius muscle by shutting down a motor nerve associated with the gastrocnemius muscle, not invading or damaging a healthy muscular tissue or gastrocnemius muscle.

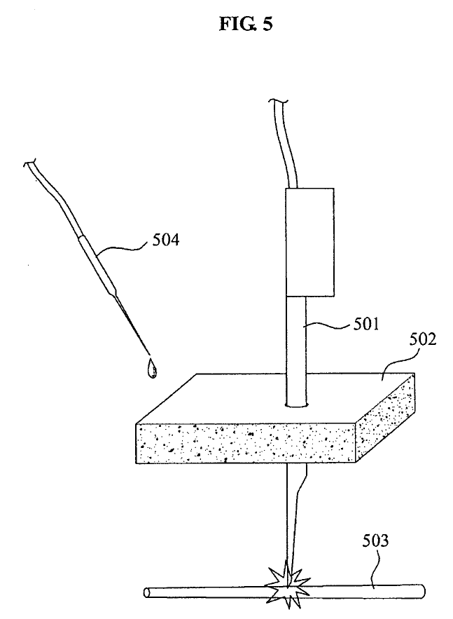

FIG. 5 is a diagram illustrating operation of shutting down of a motor nerve according to example embodiments.

As illustrated in FIG. 5, a practitioner may search for a motor nerve 503 associated with a patient's gastrocnemius muscle being searched using a nerve scan apparatus, locate a tip of the probe 501 in the motor nerve 503 by penetrating an epidermis around the motor nerve 503, shut down the motor nerve 503 by stimulating the motor nerve 503 through supply of an electric signal to the probe 501 at a first stage, and apply a dissolving agent on the motor nerve 503 using a needle 504, thereby shutting down the motor nerve 503 at a second stage. Here, the practitioner may search for the motor nerve 503 using both the nerve scan apparatus and the probe 501.

A portion of the motor nerve 503 or entire motor nerve 503 may be thinner permanently or temporarily through the first stage and through the second stage of the shutting down, consequently the gastrocnemius muscle of the patient may be thinner due to paralysis.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A surgical method for gastrocnemius muscle reduction, the method comprising:
detecting nerve line information associated with a gastrocnemius muscle of a patient using a nerve scan apparatus;
searching for a motor nerve associated with the gastrocnemius muscle based on the nerve line information;
inserting a probe by penetrating an epidermis around the motor nerve and locating a tip of the probe in the motor nerve; and
shutting down the motor nerve by stimulating the motor nerve through supply of an electric signal to the probe.

2. The method of claim 1, wherein the motor nerve is a tibial nerve.

3. The method of claim 1, wherein the gastrocnemius muscle comprises a medial gastrocnemius muscle and a lateral gastrocnemius muscle.

4. The method of claim 1, wherein the motor nerve is either a nerve to a medial gastrocnemius muscle branched from the tibial nerve or a nerve to a lateral gastrocnemius muscle.

5. The method of claim 4, wherein the searching for the motor nerve associated with the gastrocnemius muscle based on the nerve line information searches for a branch point of the tibial nerve and a common peroneal nerve, and searches for a nerve to the medial gastrocnemius muscle and a nerve to the lateral gastrocnemius muscle, branched from the tibial nerve.

6. The method of claim 1, wherein the nerve scan apparatus is an electromyogram (EMG) apparatus.

7. The method of claim 1, wherein the probe consists of an insulator which is coated on a surgical steel and coated on a surface of the surgical steel, and the tip of the probe is not coated by the insulator.

8. The method of claim 7, wherein the surgical steel includes tungsten, and the insulator is a rubber.

9. The method of claim 1, wherein the electric signal is an intermediate frequency current or a high frequency current.

10. The method of claim 1, wherein the electrical signal is transmitted to a return electrode associated with the probe via the patient.

11. The method of claim 1, wherein the shutting down of the motor nerve stimulates the motor nerve through supplying the electric signal to the probe and by applying a dissolving agent on the motor nerve using a needle, so that the motor nerve is shut down.

12. The method of claim 11, wherein the dissolving agent is ethanol or phenol.

13. The method of claim 1, wherein the searching for the motor nerve associated with the gastrocnemius muscle based on the nerve line information searches for the motor nerve associated with the gastrocnemius muscle using the probe.
